(19) United States
(12) Patent Application Publication
(10) Pub. No.: US 2026/0272490 A1
(43) Pub. Date: Sep. 17, 2026

(54) SURGICAL INSTRUMENT

(71) Applicant: EFI Holding B.V., Maastricht (NL)

(72) Inventor: Michel Petronella Hubertus Vleugels, Maastricht (NL)

(21) Appl. No.: 19/480,196

(22) PCT Filed: May 1, 2024
(86) PCT No.: PCT/EP2024/061996

§ 371 Date: Oct. 30, 2025

(30) Foreign Application Priority Data
May 1, 2023 (NL) 2034743

(51) Int. Cl. A61B 17/29; A61B 17/00; A61B 90/00

(52) U.S. Cl. CPC A61B 17/2909; A61B 90/06; A61B 2017/00367; A61B 2017/0046; A61B 2017/2903; A61B 2017/2925; A61B 2090/064; A61B 2562/0266

(57) ABSTRACT

A surgical instrument includes a shaft assembly operating device, for example a hand grip and a shaft assembly releasably couplable to the shaft assembly operating device. The shaft assembly includes a shaft and an actuation rod arranged in a longitudinal channel of the shaft. The shaft assembly operating device includes a shaft assembly coupling device to couple the shaft assembly to the shaft assembly operating device, where the shaft assembly coupling device includes a first coupling element to couple the proximal end of the shaft and a second coupling element to couple the proximal end of the actuation rod. The first coupling element and the second coupling element are movable by a single actuation from a shaft locking position to a shaft non-locking position and from an actuation rod locking position to an actuation rod non-locking position, respectively.

### CROSS-REFERENCE TO RELATED APPLICATIONS

This application is the National Stage of International Application No. PCT/EP2024/061996, filed May 1, 2024, which claims the benefit of Netherlands Application No. 2034743, filed May 1, 2023, the contents of which is incorporated by reference herein.

### FIELD OF THE INVENTION

The present invention relates to a surgical instrument. In particular, the present invention relates to a surgical instrument comprising a hand grip and a shaft assembly releasably coupled to the hand grip.

### BACKGROUND OF THE INVENTION

GB 2590181 discloses a surgical instrument comprising a hand grip and a shaft assembly. The shaft assembly comprises a shaft and an actuation rod that are provided to hold and control a working element, for example a forceps, at the distal end of the surgical instrument. To clean and sterilize the shaft and the actuation rod after use, the shaft assembly is releasably coupled to the hand grip. To couple the shaft assembly to the hand grip, the hand grip is provided with a first coupling element to couple the shaft to a fixed part of the hand grip and a second coupling element to couple the actuation rod to a movable trigger of the hand grip.

Coupling of the shaft assembly to the hand grip of the surgical instrument of GB 2590181 comprises coupling the shaft to the hand grip by movement of the first coupling element and separately coupling the actuation rod to the trigger of the hand grip by placing a head of the actuation rod in coupling engagement with the second coupling element, while the trigger is in a coupling position. Correspondingly to release the shaft assembly from the hand grip, the first coupling element must be actuated to release the shaft and the trigger must be arranged in the coupling position to release the actuation rod from the second coupling element. Due to its construction, the coupling of the shaft assembly to the hand grip according to GB 2590181 is less suitable for hand grips with a pistol type hand grip.

### OBJECT OF THE INVENTION

It is an object of the present invention to provide a surgical instrument that has an improved shaft assembly coupling device to couple the shaft assembly of a surgical instrument to the associated hand grip. It is another object of the invention to provide a shaft assembly coupling device that is suitable to couple a shaft assembly to a pistol type hand grip.

### SUMMARY OF THE INVENTION

The present invention provides a surgical instrument, for example a surgical instrument for minimally invasive surgery, having the features as described herein.

The surgical instrument of the invention comprises a shaft assembly operating device, for example a hand grip, with a shaft assembly coupling device to couple the shaft assembly to the shaft assembly operating device. The shaft assembly coupling device comprises a first coupling element to couple the proximal end of the shaft to the shaft assembly operating device and a second coupling element to couple the proximal end of the actuation rod to the shaft assembly operating device.

The first coupling element is movable between a shaft locking position in which the proximal end of the shaft is locked and a shaft non-locking position in which the proximal end of the shaft is releasable from the shaft assembly operating device. Similarly, the second coupling element is movable between an actuation rod locking position in which the proximal end of the actuation rod is locked, and an actuation rod non-locking position in which the proximal end of the actuation rod is releasable from the shaft assembly operating device.

According to the invention the first coupling element and the second coupling element are movable by a single actuation from the shaft locking position to the shaft non-locking position and from the actuation rod locking position to the actuation rod non-locking position, respectively.

This means that both the first coupling element and the second coupling element can actively be moved from the respective locking positions to the non-locking positions by a single actuation by a user. This active movement of both the first coupling element and the second coupling element from the shaft locking position to the shaft non-locking position and from the actuation rod locking position to the actuation rod non-locking position, respectively, facilitates the coupling and/or release of the shaft assembly to/from the shaft assembly operating device.

Furthermore, the active movement of the shaft assembly coupling device is carried out as a single actuation. Only one actuation element has to be actuated to allow the coupling and/or release of the shaft assembly from the shaft assembly operating device.

In an embodiment, the shaft assembly operating device comprises a trigger device, which trigger device is movable with respect to a frame and/or housing of the shaft assembly operating device, for example the hand grip. The trigger device is operated by the user to control the position of one or more working elements at a distal end of the shaft assembly, for example to control the position of the jaw elements of a forceps construction of the surgical instrument.

In an embodiment, the first coupling element is connected to a frame or housing of the shaft assembly operating device and the second coupling element is connected to a trigger device of the shaft assembly operating device. The shaft is usually coupled to the shaft assembly operating device such that the shaft is positioned in a fixed position with respect to shaft assembly operating device. Similarly, the actuation rod is connected to the trigger device to transfer movements of the trigger device via the actuation rod to the distal end of the shaft assembly, for example to jaw elements of a forceps construction to control the relative position of the jaw elements, e.g. open or closed.

In an embodiment, the first coupling element is provided with a first engagement surface and the second coupling element is provided with a second engagement surface, wherein the first engagement surface and the second engagement surface are arranged to engage each other when one of the first coupling element and the second coupling element is moved from the shaft locking position to the shaft non-locking position or from the actuation rod locking position to the actuation rod non-locking position, respectively, to push or pull the other of the first coupling element and the second coupling element from the shaft locking position to the shaft non-locking position or from the actuation rod locking position to the actuation rod non-locking position, respectively.

By using the movement of one of the first coupling element and the second coupling element to push the other of the first coupling element and the second coupling element to a respective non-locking position both the first coupling element and the second coupling element are simultaneously moved to the non-locking position while only one of the first coupling element and the second coupling element is actually actuated. This may be advantageous as the coupling locations of the shaft and the actuation rod may move with respect to each other due to the relative position of a trigger device with respect to the position of the frame and/or housing of the shaft assembly operating device. By pushing or pulling of one of the first and second coupling element by the other of the first and second coupling element using engagement between the first and second engagement surfaces, simultaneous movement from the respective locking positions to the respective non-locking positions is facilitated while still allowing relative movement between the coupling location of the shaft and the coupling location of the actuation rod.

In an embodiment, the shaft assembly coupling device comprises a first biasing device to bias the first coupling element to the shaft locking position. To ensure that the shaft does not inadvertently release from the shaft assembly operating device, a first biasing device may be provided to bias the first coupling element in the shaft locking position. The first biasing device is for example a spring, such as a helical shaped spring.

In an embodiment, the shaft assembly coupling device comprises a second biasing device to bias the second coupling element to the actuation rod locking position. To ensure that the actuation rod does not inadvertently release from the shaft assembly operating device, a second biasing device may be provided to bias the second coupling element in the actuation rod locking position.

In an embodiment, the second biasing device may comprise a lever with two arms, a free end of a first arm of the two arms being connected to a spring, and a free end of a second arm of the two arms having a contact surface to engage an associated contact surface of the second coupling element. The second biasing device may be mounted on the trigger device to move together with the second coupling element when the trigger device is displaced with respect to the frame or housing of the shaft assembly operating device, for example to change the relative positions of the jaw elements of the forceps construction at the distal end of the shaft assembly. In an alternative embodiment, the second biasing device may be mounted on a frame or housing of the shaft assembly operating device.

In an embodiment, the single actuation is carried out by movement of an actuation element from a locking position to a non-locking position. The shaft assembly coupling mechanism may comprise an actuation element, for example a button-like element to be pushed by a user to carry out the single actuation of both the first coupling element and the second coupling element. The actuation element may be mounted on one of the first coupling element and the second coupling element, or the actuation element may be an integral part of the first coupling element or the second coupling element.

In an embodiment, the first coupling element or the second coupling element is the actuation element. The actuation element may be formed by the first coupling element or the second coupling element. For example, the actuation element may be integrally formed by the first coupling element. The second coupling element may in such embodiment be movable by actuation of the first coupling element, i.e. actuation of the actuation element integrated in the first coupling element may result in movement of the first coupling element from the shaft locking position to the shaft non-locking position. During this movement the first coupling element may push or pull the second coupling element from the actuation rod locking position to the actuation rod non-locking position.

In an embodiment, the proximal end of the shaft comprises a shaft head having a shaft head diameter, wherein the first coupling element comprises a shaft lock slot comprising a first end having a first width and a second end having a second width, wherein the first width is smaller than the shaft head diameter and wherein the second width is larger than the shaft head diameter. This configuration of the shaft lock slot can be used to lock the shaft head in the shaft locking position and to release the shaft head in the shaft non-locking position. In the shaft locking position, the shaft may be aligned with the first end of the shaft lock slot and the shaft head may be arranged proximal of the shaft lock slot. In this position of the shaft lock slot, the shaft cannot be pulled out of the shaft assembly coupling device as the shaft head is locked by the first end of the shaft lock slot. In the shaft non-locking position, the shaft may be aligned with the second end of the shaft lock slot. In this position of the shaft lock slot with respect to the shaft, the shaft can be moved freely in and out of the shaft assembly coupling device as movement of the shaft head is not blocked by the shaft lock slot.

Any other configuration of a proximal end of the shaft that can be locked in a shaft locking position of a first coupling element and released in a shaft non-locking position of the first coupling element may also be applied. For example, the proximal end of the shaft may be provided with a groove configured to receive, in the shaft locking position, the first coupling element to block any longitudinal movement of the shaft. In the shaft non-locking position, the first coupling element may be displaced such that the first coupling element does not extend into the groove to make coupling and release of the shaft from the shaft assembly operating device possible.

In an embodiment, the proximal end of the actuation rod comprises an actuation rod head having an actuation rod head diameter and wherein the second coupling element comprises an actuation rod lock slot comprising a first end having a first width and a second end having a second width, wherein the first width is smaller than the actuation rod head diameter and wherein the second width is larger than the actuation rod head diameter. Similar to the shaft lock slot, the actuation rod lock slot may be used to effectively lock the actuation rod head in the actuation rod locking position by the second coupling element. In the actuation rod locking position, the actuation rod may be aligned with the first end of the actuation rod lock slot and the actuation rod head may be arranged proximal of the actuation rod lock slot. In this position of the actuation rod lock slot, the actuation rod cannot be pulled out of the shaft assembly coupling device as the actuation rod head is locked by the first end of the actuation rod lock slot. In the actuation rod non-locking position, the actuation rod may be aligned with the second end of the actuation rod lock slot. In this position of the actuation rod lock slot with respect to the actuation rod, the actuation rod can be moved freely in and out of the shaft assembly coupling device as movement of the actuation rod head is not blocked by the actuation rod lock slot.

Any other configuration of a proximal end of the actuation rod that can be locked in an actuation rod locking position of a second coupling element and released in an actuation rod non-locking position of the second coupling element may also be applied. For example, the proximal end of the actuation rod may be provided with a groove configured to receive, in the actuation rod locking position, the second coupling element to block any longitudinal movement of the actuation rod. In the actuation rod non-locking position, the second coupling element may be displaced such that the second coupling element does not extend into the groove to make coupling and release of the actuation rod from the shaft assembly operating device possible.

In an embodiment, the first coupling element comprises two cam extensions and the second coupling element comprises two pin like extensions extending at opposite sides of a main body of the second coupling element, wherein the cam extensions are arranged to engage the pin like extensions when the first coupling element is moved from the shaft locking position to the shaft non-locking position to simultaneously move the second coupling element from the actuation rod locking position to the actuation rod non-locking position.

In this embodiment, the cam extensions provide first engagement surfaces and the pin like extensions provide second engagement surfaces, wherein the first engagement surfaces and the second engagement surfaces are arranged to engage each other when the first coupling element is moved from the shaft locking position to the shaft non-locking position with the result that during movement of the first coupling element from the shaft locking position to the shaft non-locking position, the second coupling element is moved from the actuation rod locking position to the actuation rod non-locking position.

In an embodiment, the surgical instrument comprises a first coupling element guide to guide movement of the first coupling element between the shaft locking position and the shaft non-locking position. The first coupling element guide may be any construction to guide movement of the first coupling element between the shaft locking position to the shaft non-locking position.

The first coupling element guide may be formed by or mounted on a frame and/or housing of the shaft assembly operating device. For example, the first coupling element guide may comprise guiding slots provided in a frame of the shaft assembly operating device that cooperate with guiding pins mounted on the first coupling element. The guiding slots may extend parallel to the desired direction of movement of the first coupling element from the shaft locking position and the shaft non-locking position to enable a linear movement of the first coupling element in this direction.

In an embodiment, the surgical instrument comprises a second coupling element guide to guide movement of the second coupling element between the actuation rod locking position and the actuation rod non-locking position. The second coupling element guide guides movement of the second coupling element between the actuation rod locking position and the actuation rod non-locking position. The second coupling element guide may be mounted on the trigger device, such that the second coupling element guide moves together with an actuation movement of the trigger device. The second coupling element guide may for example define a channel having a diameter that substantially corresponds or is slightly larger than an outer diameter of the second coupling element such that the second coupling element may slide in a linear movement through the channel.

In an embodiment the second coupling element may have a substantially cylindrical shape and the second coupling element guide may define an internal channel that is designed to receive and guide the substantially cylindrical shape of the second coupling element.

In an embodiment, the second coupling element guide defines a channel through which the second coupling element may slide, wherein an inner surface of the channel comprises a recess to receive a proximal end surface of the actuation rod. The recess facilitates that the actuation rod may be arranged at a correct position in the shaft assembly coupling device for coupling of the actuation rod by the second coupling element. The recess may also improve the locking of the actuation rod by the second coupling element.

In an embodiment, the first coupling element is movable from the shaft locking position to the shaft non-locking position with a first linear movement in a first movement direction and the second coupling element is movable from the actuation rod locking position to the actuation rod non-locking position with a second linear movement in a second movement direction.

It is advantageous that the first coupling element and the second coupling element are both movable in a linear direction in order to move by a single actuation the first coupling element and the second coupling element from the shaft locking position to the shaft non-locking position and from the actuation rod locking position to the actuation rod non-locking position, respectively.

In an embodiment, an angle between the first movement direction and the second movement direction is less than 45 degrees, for example less than 30 degrees to facilitate simultaneous movement of the first coupling element and the second coupling element by a single actuation.

In an embodiment, the shaft assembly operating device comprises a first optical fibre part and a first connector connected to the first optical fibre part and the shaft assembly comprises a second optical fibre part and a second connector connected to the second optical fibre part, wherein during coupling of the shaft assembly and the shaft assembly operating device, the first connector and the second connector are connected to each other to optically couple the first optical fibre part and the second optical fibre part.

An optical fibre may be used to transfer an optical signal between the shaft assembly and the shaft assembly operating device. For example, an optical sensor arranged in or combined with an optical fibre, for example a Fibre Bragg grating (FBG), may be used to measure a force exerted on jaw elements at the distal end of the shaft assembly. To ensure a proper transfer of the optical signal between the shaft assembly and the shaft assembly operating device, the first connector and the second connector may be connected to each other during coupling of the shaft assembly to the shaft assembly operating device to optically couple the first optical fibre part shaft assembly operating device and the second optical fibre part of the shaft assembly.

The coupling between the shaft assembly and the shaft assembly operating device as provided by the shaft assembly coupling device described herein, is suitable to be used to provide such coupling of the first connector and the second connector. This coupling may ensure that the optical end surfaces of the first optical fibre part and the second optical fibre part are properly placed against each other to form an aligned optical fibre to transfer the optical signal.

Further details of an embodiment of an optical connection using a first connector and a second connector to couple the first optical fibre part and the second optical fibre part are disclosed in WO2020/101496A1.

In an embodiment, the shaft assembly operating device is a hand grip, for example a pistol type hand grip. In prior art, different grips are proposed to hold and operate a surgical instrument, in particular a surgical instrument for minimally invasive surgery. One of these types of grips is a pistol type grip. In such surgical instrument, the surgical instrument is held and operated similar to a pistol, in which the hand grip is held by the palm of a hand and the trigger is operated by an index finger and/or middle finger of the same hand. The shaft assembly coupling device as described herein is in particular suitable to be used in a pistol type hand grip, but may also be applied in other configurations of a surgical instrument. More generally, the shaft assembly coupling device is useful in a surgical instrument in which it is difficult or unpractical to require a separate action for coupling of the actuation rod to the hand grip, in particular to the trigger device of the hand grip of the surgical instrument.

In other embodiments, the shaft assembly operating device may be an actuation system of a surgical robot system. The shaft assembly operating device may for example be a robot arm configured to be coupled to the shaft assembly to support and actuate the shaft assembly.

The shaft assembly operating device may also be any other device configured to be coupled to the shaft assembly to operate the shaft assembly.

The combination of shaft assembly operating device and shaft assembly using a shaft assembly coupling device to couple the shaft assembly to the shaft assembly operating device as described herein may also be used in any other suitable instrument, for example in a hand-held or robotic industrial instrument.

### BRIEF DESCRIPTION OF THE DRAWINGS

Embodiments of the invention will be described below with reference to the accompanying drawings, in which:

FIG. 1 shows a surgical instrument having a hand grip and a shaft assembly releasably coupled to the hand grip;

FIGS. 2 and 3 show cross sections of a releasable coupling of the shaft assembly to the hand grip;

FIGS. 4 and 5 show the proximal end of the actuation rod, the second coupling element and the second biasing device in more detail; and

FIGS. 6 and 7 show the cross sections of FIGS. 2 and 3 with the contour of the first coupling element.

### DETAILED DESCRIPTION OF THE INVENTION

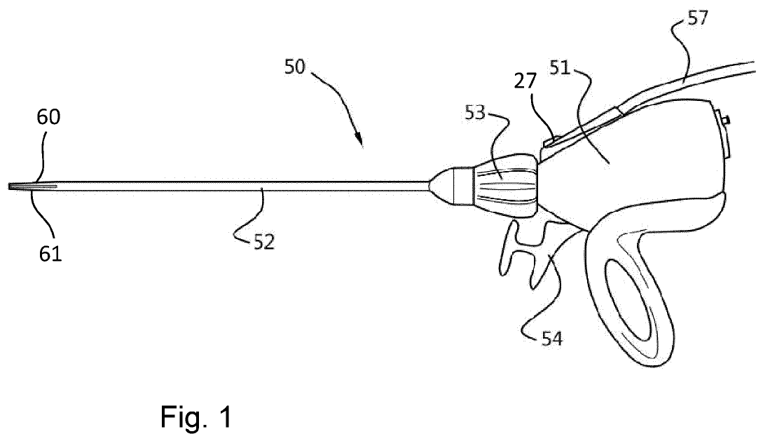

FIG. 1 shows a surgical instrument 50 for minimally invasive surgery. The surgical instrument 50 comprises a hand grip 51 and a shaft assembly 52. The hand grip 51 comprises a frame and a housing mounted on the frame. The shaft assembly 52 is releasably on the hand grip 51. A release button 27 is provided to release the shaft assembly 52 from the hand grip.

Further, the shaft assembly 52 is rotatable about its longitudinal axis with respect to the handle grip 51. This allows different rotational positions of the shaft assembly 52 with respect to the hand grip 51 of the surgical instrument 50. A rotation knob 53 is provided to manually set a rotation position of the shaft assembly 52 with respect to the hand grip 51.

The rotation of the shaft about its longitudinal axis may for example be in the range of 300 degrees to 360 degrees, for example in the range of 160 degrees to 170 degrees in both rotation directions from a middle rotation position of the shaft. One or more stop elements may be provided to limit the range of rotation of the shaft assembly 52 with respect to the hand grip 51.

A trigger device 54 is provided to operate jaw elements 60, 61 of a forceps construction arranged at a distal end of the shaft assembly 52. The trigger device 54 is rotatably mounted in the hand grip 51 of the surgical instrument 50 to control the jaw elements 60, 61 by movements of the trigger device 54.

A sensor may be arranged to measure a force exerted on the jaw elements 60, 61 of the forceps construction. The sensor may comprise a fibre Bragg grating FBG provided in an optical fibre arranged in the shaft assembly 52. A feedback actuator may be provided in the hand grip 51 to exert a feedback force on the trigger device 54 on the basis of a sensor signal based on the sensor signal obtained from the FBG, or another sensor.

A cable 57 is connected to the hand grip 51. The cable 57 may guide the optical fibre from the hand grip 51 to an interrogator device (not shown) arranged at a separate location. The interrogator device is configured to interrogate the FBG provided in the optical fibre and to provide a sensor signal representative for the force exerted on the jaw elements 60, 61 of the forceps construction.

The surgical instrument may further comprise a controller, wherein the controller is arranged to control the feedback actuator on the basis of the sensor signal. The controller may be part of the hand grip 51 of the surgical instrument 50. In this embodiment, the sensor signal obtained by the interrogator device is guided through the cable 57 back to the hand grip 51. In another embodiment, the controller may be provided as a separate device, or for example integrated with the interrogator device. In this embodiment the cable 57 is used to guide a control signal of the controller to the feedback actuator. In yet an alternative embodiment, the controller and the interrogator device may be integrated in the hand grip 51 of the surgical instrument 50.

Further details of an embodiment of a surgical instrument in which the shaft assembly coupling device disclosed herein can be applied, are disclosed in WO2020/101496A1, the contents of which are herein incorporated by reference.

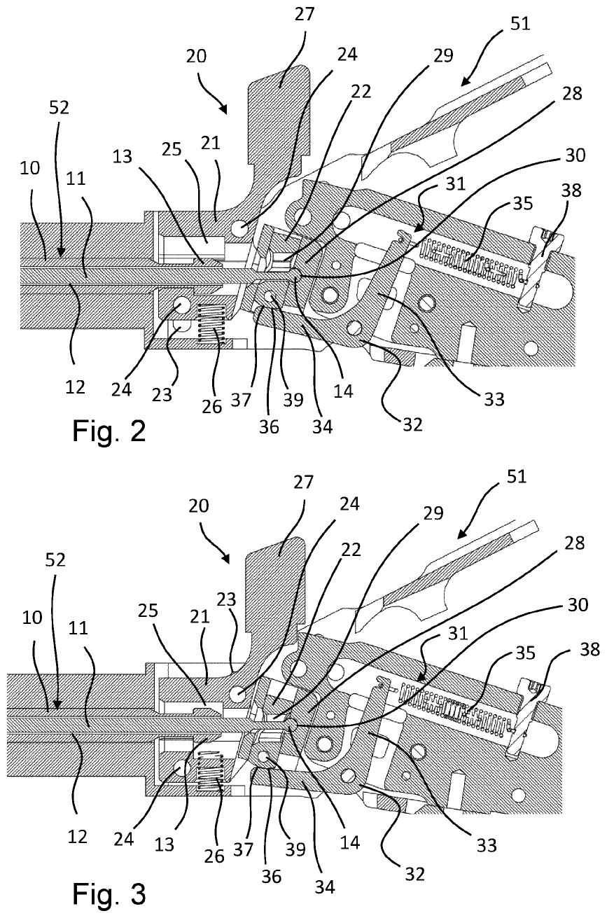

FIGS. 2 and 3 show cross-sections of the releasable coupling of the shaft assembly 52 to the hand grip 51 that can be applied in the surgical instrument 50 of FIG. 1.

The shaft assembly 52 comprises a shaft 10 and an actuation rod 11. The shaft 10 has a longitudinal channel 12 in which the actuation rod 11 is arranged. The shaft 10 has an elongated shape comprising a distal end and a proximal end. At the distal end of the shaft 10, the forceps construction having jaw elements 60, 61 is mounted. The proximal end of the shaft comprises a shaft head 13. The shaft head 13 is a thickened portion of the shaft 10 used to couple the shaft 10 to the hand grip 10 as described hereinafter.

The actuation rod 11 also comprises an elongated shape having a proximal end and a distal end. The proximal end of the actuation rod 11 is operatively connected to the trigger device 54. The distal end of the actuation rod 11 is operatively connected to the jaw elements 60, 61 of the forceps construction to transfer movement/forces from the trigger device 54 to the forceps construction. The proximal end of the actuation rod comprises an actuation rod head 14. Similar to the shaft head 13 is the actuation rod head 14 provided as a thickened portion of the actuation rod 11 used to couple the actuation rod 11 to the hand grip 51, in particular the trigger device 54, as described hereinafter.

The hand grip 51 comprises a shaft assembly coupling device 20 to couple the shaft assembly 52 to the hand grip 51. The shaft assembly coupling device 20 comprises a first coupling element 21 and a second coupling element 22.

The first coupling element 21 is arranged to couple the proximal end of the shaft 10 to the hand grip 51, while the second coupling element 22 is arranged to couple the proximal end of the actuation rod 11 to the trigger device 54 of the hand grip 51.

The first coupling element 21 is movable between a shaft locking position (shown in FIG. 2) in which the proximal end of the shaft 10 is locked by the first coupling element 21, and a shaft non-locking position (shown in FIG. 3) in which the proximal end of the shaft 10 is releasable from the hand grip 51.

The movement of the first coupling element 21 between the shaft locking position and the shaft non-locking position is guided by a first coupling element guide to guide the movement of the first coupling element. This first coupling element guide is formed by guiding slots 23 arranged in the frame of the hand grip 51 and extending in a direction of movement of the first coupling element 21. The first coupling element 21 comprises guiding pins 24 that extend perpendicular to the direction of movement of the first coupling element 21 (and perpendicular to the plane of the drawing) and into the guiding slots 23. The guiding pins 24 extend at opposite sides of the first coupling element 21 (only one side shown) to cooperate with the guiding slots 23 that are arranged at opposite sides of the first coupling element 21 in the frame of the hand grip 51.

The shaft head 13 at the proximal end of the shaft 10 comprises a shaft head diameter. The first coupling element 21 comprises a shaft lock slot 25 comprising a first end having a first width and a second end having a second width, wherein the first width is smaller than the shaft head diameter and wherein the second width is larger than the shaft head diameter. In the shaft locking position, shown in FIG. 2, the first end of the shaft lock slot 25 is aligned with the shaft 10, such that the shaft head 13 is locked in the shaft lock slot 25. In the shaft non-locking position, shown in FIG. 3, the second end of the shaft lock slot 25 is aligned with the shaft 10, such that the shaft head 13 is not locked in the shaft lock slot 25 and can be freely moved in and out of the shaft lock slot 25.

A first biasing device 26 is provided to bias the first coupling element 13 into the shaft locking position, such that the shaft head 13, when placed through the shaft lock slot 25 can only be moved out of the shaft lock slot 25, if the first coupling element 21 is actively moved from the shaft locking position to the shaft non-locking position against a biasing force of the first biasing device 26. The release button 27, which is a fixed on the first coupling element 22 or an integral part thereof, can be pushed by a user to move the first coupling element 21, against the biasing force of the first biasing device 26, from the shaft locking position to the shaft non-locking position. The first biasing device 26 comprises a helix shaped spring element.

The second coupling element 22 is arranged to couple the proximal end of the actuation rod 11 to the hand grip 51, in particular to the trigger device 54 of the hand grip 51.

The second coupling element 22 is movable between an actuation rod locking position (shown in FIG. 2) in which the proximal end of the actuation rod 11 is locked by the second coupling element 22, and an actuation rod non-locking position (shown in FIG. 3) in which the proximal end of the actuation rod 11 is releasable from the hand grip 51, in particular the trigger device 54 of the hand grip 51. The proximal end of the actuation rod 11 may be connected to any part of the trigger device 54 that is capable of transferring movement of the trigger device 54 to the actuation rod 11.

The movement of the second coupling element 22 between the actuation rod locking position and the actuation rod non-locking position is guided by a second coupling element guide 28. The second coupling element guide 28 is mounted on the trigger device 54 so that the second coupling element guide 28 will move together with any movement of the trigger device 54.

The second coupling element guide 28 defines an internal channel having an inner diameter that substantially corresponds or is slightly larger than an outer diameter of the second coupling element 22 such that the second coupling element 22 can slide in a linear movement in the internal channel of the second coupling element guide 28. The internal channel may be an open channel, i.e. a channel having side openings, as long as the second coupling element 22 may be guided by the shape of the channel.

The actuation rod head 14 at the proximal end of the actuation rod 11 comprises an actuation rod head diameter. The second coupling element 22 comprises an actuation rod lock slot 29 comprising a first end having a first width and a second end having a second width, wherein the first width is smaller than the actuation rod head diameter and wherein the second width is larger than the actuation rod head diameter. In the actuation rod locking position, shown in FIG. 2, the first end of the actuation rod lock slot 29 is aligned with the actuation rod 11, such that the actuation rod head 14 is locked in the actuation rod lock slot 29. In the shaft non-locking position, shown in FIG. 3, the second end of the actuation rod lock slot 29 is aligned with the actuation rod 11, such that the actuation rod head 14 is not locked in the actuation rod lock slot 29 and can be freely moved in and out of the actuation rod lock slot 29.

The second coupling element guide 28 comprises on the surface of the internal channel a recess 30 arranged to receive the end surface of the actuation rod head 14 when the actuation rod 11 is brought into the hand grip 51. The recess 30 facilitates that the actuation rod 11 is arranged at a correct position in the shaft assembly coupling device 20 for coupling of the shaft assembly 52. This position is of importance since the locking of the actuation rod in the correct position also may ensure that movements of the trigger device 54 are accurately transferred to the jaw elements 60, 61 of the forceps construction. The recess 30 may also improve the locking of the actuation rod head 14 by the second coupling element 22.

A second biasing device 31 is provided to bias the second coupling element 22 to the actuation rod locking position. The second biasing device comprises a lever 32 with a first arm 33 and a second arm 34. The lever 32 can pivot about a pivot point at the origin of the first arm 33 and the second arm 34. A free end of the first arm 33 of the lever 32 is connected to a spring 35. A free end of the second arm 34 has a first contact surface 36 to engage an associated second contact surface 37 of the second coupling element 22.

The spring 35 is arranged between the free end of the first arm 33 and a bolt 38. The spring 35 exerts a pulling force on the free end of the first arm 33 to bias the lever 32 in a clockwise direction, therewith pushing the second coupling element 22 by the contact between the first contact surface 36 and the second contact surface 37 in the direction of the actuation rod locking position shown in FIG. 2.

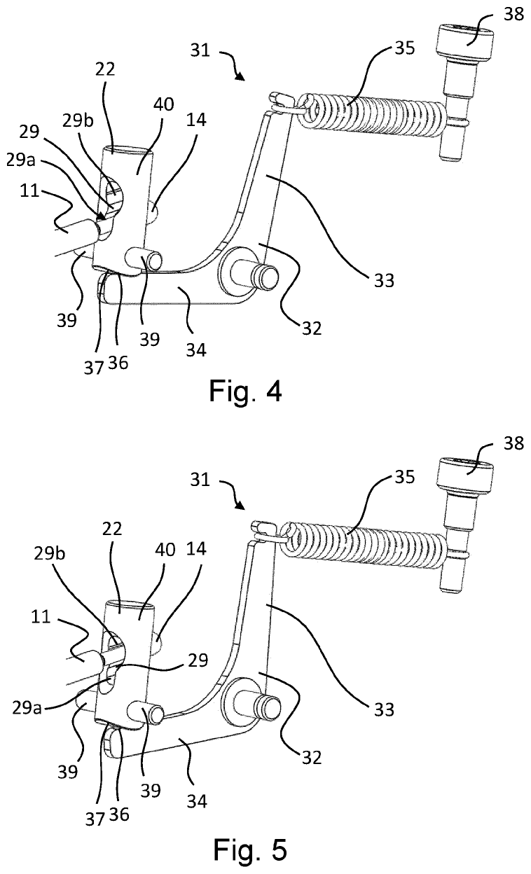

FIGS. 4 and 5 show the proximal end of the actuation rod 14, the second coupling element 22 and the second biasing device 31 in more detail. FIG. 4 shows the second coupling element 22 and the second biasing device 31 in the actuation rod locking position in which the first end 29a of the actuation rod lock slot 29 is aligned with the actuation rod 11 and FIG. 5 shows the second coupling element 22 and the second biasing device 31 in the actuation rod non-locking position in which the second end 29b of the actuation rod lock slot 29 is aligned with the actuation rod 11.

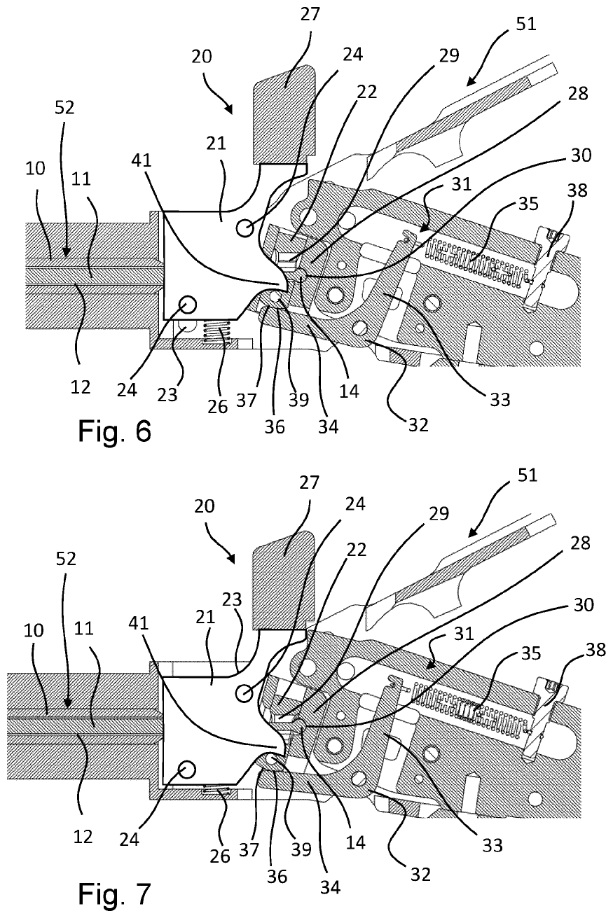

The second coupling element 22 comprises two pin like extensions 39 extending at opposite sides of a main body 40 of the second coupling element 22. These two pin like extensions 39 can be used to push the second coupling element 22, against the biasing force of the second biasing device 31, from the actuation rod locking position to the actuation rod non-locking position. The pin like extensions 39 on the second coupling element 22 are provided to cooperate with cam extensions 41 (see FIGS. 6 and 7) provided on the first coupling element 21 in order to move the second coupling element 22 from the actuation rod locking position to the actuation rod non-locking position simultaneously with a movement of the first coupling element 21 from the shaft locking position to the shaft non-locking position.

FIGS. 6 and 7 show the cross sections of FIGS. 2 and 3 with a full contour of the first coupling element 22. This full contour of the first coupling element 21 can be found in planes parallel and spaced from the plane of the drawing at equal distance above and under the drawing. The cam extensions 41 are arranged to cooperate with the pin like extensions 39 of the second coupling element 22. In this embodiment, the first coupling element 21 is provided with a first engagement surface on each of the cam extensions 41 and the second coupling element 22 is provided with a second engagement surface on each of the pin like extensions 39. Each first engagement surface on a respective cam extension 41 is arranged to engage an associated second engagement surface on a pin like extension 39 when the first coupling element 22 is moved from the shaft locking position to the shaft non-locking position by pushing on the release button 27. The engagement between the first engagement surface and the second engagement surface will simultaneously with the movement of the first coupling element 21 move the second coupling element 22 from the actuation rod locking position to the actuation rod non-locking position.

Thus, this arrangement of the shaft assembly coupling device facilitates that the first coupling element 21 and the second coupling element 22 are movable from the shaft locking position to the shaft non-locking position and from the actuation rod locking position to the actuation rod non-locking position, respectively, by a single actuation of the release button 27 Once the first coupling element 21 is positioned in the shaft non-locking position and the second coupling element 22 is positioned in the actuation rod non-locking position, the shaft assembly 52 can be taken out of the hand grip 51. This shaft assembly coupling device 20 therefore enables a reliable and user-friendly release of the shaft assembly 52 from the hand grip 51.

The shaft assembly coupling device 20 may be configured such that when the shaft 10 and actuation rod 11 are pushed into the hand grip 51, the shaft 10 will push, for example by using slanted surfaces, the first coupling element 21 towards the shaft non-locking position to allow the shaft head 13 to pass the shaft lock slot 25. As soon as the shaft head 13 is pushed through the shaft lock slot 25, the first coupling element 21 may be pushed back by the first biasing device 26 into the shaft locking position, in which the shaft head 13 is locked by the first coupling element 21. Similarly, when the shaft 10 and actuation rod 11 are pushed into the hand grip 51, the actuation rod 11 may push, for example by using slanted surfaces, the second coupling element 22 towards the actuation rod non-locking position to allow the actuation rod head 14 to pass the actuation rod lock slot 29. As soon as the actuation rod head 14 is pushed through the actuation rod lock slot 29, the second coupling element 22 may be pushed back by the second biasing device 31 into the actuation rod locking position, in which the actuation rod head 13 is locked by the second coupling element 22. Such configuration would allow to couple the shaft assembly 52 to the hand grip 51 by pushing the shaft 10 and actuation rod 11 into the hand grip 51 without the need to also push down the release button 27.

To facilitate the coupling of the shaft assembly 52 to the hand grip 51, the user may while moving the shaft 10 and the actuation rod 11 into the hand grip 51, push the release button 27 to move the first coupling element 21 from the shaft locking position to the shaft non-locking position and, therewith, the second coupling element 22 from the actuation rod locking position to the actuation rod non-locking position.

In an alternative configuration, it is possible that the shaft assembly 52 can only be coupled to the hand grip 51 when the release button 27 is pushed to move the first coupling element 21 from the shaft locking position to the shaft non-locking position and the second coupling element 22 from the actuation rod locking position to the actuation rod non-locking position.

In both configurations, it is required that the release button 27 is pushed to move the first coupling element 21 from the shaft locking position to the shaft non-locking position and therewith the second coupling element 22 from the actuation rod locking position to the actuation rod non-locking position in order to make release of the shaft assembly 52 from the hand grip 51 possible. It is undesirable that the shaft assembly 52 may release from the hand grip 51 without active actuation of the release button 27.

Further, in both configurations, the coupling of the actuation rod 11 to and release of the actuation rod 11 from the second coupling element 22 may be independent of the operating position of the trigger device 54, i.e. the coupling and/or release can be carried out independent of a position of the trigger device 54 with respect to the frame/housing of the hand grip 51. In other embodiments, the coupling of the actuation rod 11 to and release of the actuation rod 11 from the second coupling element 22, can only be carried out in a specific position or range of positions of the trigger device 54.

Hereinabove, an embodiment of a shaft assembly coupling device 20 has been described in relation to a surgical instrument as disclosed in WO2020/101496A1. The same or a similar construction of a shaft assembly coupling device may be applied in any other surgical instrument, for example a surgical instrument for minimally invasive surgery, or any non-surgical instrument, in which a shaft assembly comprising a shaft and an actuation rod has to be releasably coupled to a hand grip. The shaft assembly coupling device may be in particular beneficial in a pistol type surgical instrument, or more generally, in a surgical instrument in which it is difficult or unpractical to require a separate action for coupling of the actuation rod to the hand grip, in particular to the trigger device of the hand grip of the surgical instrument.

1. A surgical instrument, comprising:
a shaft assembly operating device, for example a hand grip, and
a shaft assembly, that is releasably couplable to the shaft assembly operating device, the shaft assembly comprising:
a shaft, the shaft having a longitudinal channel, the shaft having a proximal end and a distal end,
an actuation rod arranged in the longitudinal channel, the actuation rod having a proximal end and a distal end,
wherein the shaft assembly operating device comprises a shaft assembly coupling device to couple the shaft assembly to the shaft assembly operating device, wherein the shaft assembly coupling device comprises:
a first coupling element to couple the proximal end of the shaft, wherein the first coupling element is movable between a shaft locking position in which the proximal end of the shaft is locked and a shaft non-locking position in which the proximal end of the shaft is releasable from the shaft assembly operating device, and
a second coupling element to couple the proximal end of the actuation rod, wherein the second coupling element is movable between an actuation rod locking position in which the proximal end of the actuation rod is locked, and an actuation rod non-locking position in which the proximal end of the actuation rod is releasable from the shaft assembly operating device,
wherein the first coupling element and the second coupling element are movable by a single actuation from the shaft locking position to the shaft non-locking position and from the actuation rod locking position to the actuation rod non-locking position, respectively.

2. The surgical instrument of claim 1, wherein the first coupling element is provided with a first engagement surface and the second coupling element is provided with a second engagement surface, wherein the first engagement surface and the second engagement surface are arranged to engage each other when one of the first coupling element and the second coupling element is moved from the shaft locking position to the shaft non-locking position or from the actuation rod locking position to the actuation rod non-locking position, respectively, to push or pull the other of the first coupling element and the second coupling element from the shaft locking position to the shaft non-locking position or from the actuation rod locking position to the actuation rod non-locking position, respectively.

3. The surgical instrument of claim 1, wherein the shaft assembly coupling device comprises a first biasing device to bias the first coupling element to the shaft locking position.

4. The surgical instrument of claim 1, wherein the shaft assembly coupling device comprises a second biasing device to bias the second coupling element to the actuation rod locking position.

5. The surgical instrument of claim 4, wherein the second biasing device comprises a lever with two arms, a free end of a first arm of the two arms being connected to a spring, and a free end of a second arm of the two arms having a contact surface to engage an associated contact surface of the second coupling element.

6. The surgical instrument of any of claim 1, wherein the single actuation is carried out by movement of an actuation element from a locking position to a non-locking position.

7. The surgical instrument of claim 6, wherein the first coupling element or the second coupling element is the actuation element.

8. The surgical instrument of claim 6, wherein the actuation element comprises a button-like element to be pushed by the user.

9. The surgical instrument of any of claim 1, wherein the proximal end of the shaft comprises a shaft head having a shaft head diameter and wherein the first coupling element comprises a shaft lock slot comprising a first end having a first width and a second end having a second width, wherein the first width is smaller than the shaft head diameter and wherein the second width is larger than the shaft head diameter.

10. The surgical instrument of any of claim 1, wherein the proximal end of the actuation rod comprises an actuation rod head having an actuation rod head diameter and wherein the second coupling element comprises an actuation rod lock slot comprising a first end having a first width and a second end having a second width, wherein the first width is smaller than the actuation rod head diameter and wherein the second width is larger than the actuation rod head diameter.

11. The surgical instrument of claim 1, wherein the first coupling element comprises two cam extensions and wherein the second coupling element comprises two pin like extensions extending at opposite sides of a main body of the second coupling element, wherein the cam extensions are arranged to engage the pin like extensions when the first coupling element is moved from the shaft locking position to the shaft non-locking position to simultaneously move the second coupling element from the actuation rod locking position to the actuation rod non-locking position.

12. The surgical instrument of claim 1, wherein the surgical instrument comprises a first coupling element guide to guide movement of the first coupling element between the shaft locking position and the shaft non-locking position.

13. The surgical instrument of claim 1, wherein the surgical instrument comprises a second coupling element guide to guide movement of the second coupling element between the actuation rod locking position and the actuation rod non-locking position.

14. The surgical instrument of claim 13, wherein the second coupling element guide defines a channel through which the second coupling element may slide, wherein an inner surface of the channel comprises a recess to receive a proximal end surface of the actuation rod.

15. The surgical instrument of claim 1, wherein the first coupling element is movable from the shaft locking position to the shaft non-locking position with a first linear movement in a first movement direction and wherein the second coupling element is movable from the actuation rod locking position to the actuation rod non-locking position with a second linear movement in a second movement direction.

16. The surgical instrument of claim 15, wherein an angle between the first movement direction and the second movement direction is less than 45 degrees, for example less than 30 degrees.

17. The surgical instrument of claim 1, wherein the shaft assembly operating device comprises a first optical fibre part and a first connector connected to the first optical fibre part and wherein the shaft assembly comprises a second optical fibre part and a second connector connected to the second optical fibre part, wherein during coupling of the shaft assembly and the shaft assembly operating device, the first connector and the second connector are connected to each other to optically couple the first optical fibre part and the second optical fibre part.

18. The surgical instrument of claim 1, wherein the shaft assembly operating device is a hand grip, for example a pistol type hand grip.
